(19) United States
(12) Patent Application Publication
(10) Pub. No.: US 2006/0188989 A1
(43) Pub. Date: Aug. 24, 2006

(54) Inhibition of E3-ubiquitin ligase HAKAI for treatment of proliferative disorders

(75) Inventors: Annette O. Walter, San Carlos, CA (US); Christoph Reinhard, Alameda, CA (US)

Correspondence Address: Chiron Corporation;Intellectual Property - R440, P.O. Box 8097, Emeryville, CA, 94662-8097, US

(73) Assignee: Chiron Corporation, Emeryville, CA (US)

(21) Appl. No.: 11/401,896

(22) Filed: Apr. 12, 2006

Related U.S. Application Data
(62) Division of application No. 10/754,643, filed on Jan. 12, 2004.
(60) Provisional application No. 60/440,030, filed on Jan. 15, 2003.

(51) Int. Cl. A61K 48/00; C12N 15/87

(52) U.S. Cl. 435/455; 514/44

(57) ABSTRACT

Human HAKAI (hsHAKAI), an E3-ubiquitin ligase, can be inhibited to treat proliferative disorders, such as cancers, dysplasias and hyperplasias. Effective levels of hsHAKAI can be inhibited, for example, using antisense oligonucleotides, ribozymes, interference RNA, and antibodies. Test compounds can be screened for binding to hsHAKAI, for disruption of hsHAKAI-E-cadherin binding, or for inhibition of hsHAKAI enzymatic activity to identify therapeutic compounds for treating proliferative disorders.

This application is a division of co-pending application Ser. No. 10/754,643 filed Jan. 12, 2004, which claims the benefit of provisional application Ser. No. 60/440,030 filed Jan. 15, 2003. Both applications are incorporated herein by reference.

### FIELD OF THE INVENTION

The invention relates to decreasing effective levels of an E3-ubiquitin ligase, hsHAKAI, to treat cancer and other proliferative disorders.

### BACKGROUND OF THE INVENTION

Tumor cells down-regulate levels of the cell-surface protein E-cadherin during the transition from an adenoma to a carcinoma. Tyrosine phosphorylated E-cadherin is ubiquitinated at the plasma membrane, inducing endocytosis. Fujita et al., Nature Cell Biol. 4, 222-31, 2002. In mice, the post-translational regulator of E-cadherin stability is the E3-ubiquitin ligase “HAKAI,” which binds to E-cadherin. Id. Mouse HAKAI is a 491 amino acid protein that resembles c-Cbl. Activation of Src results in ubiquitination of E-cadherin by HAKAI. Mutation of C109A of HAKAI, a conserved residue in its ring finger domain that is required for ubiquitin ligase activity, interfered with ubiquitination in the presence of v-Src. MDCK cells transfected with mouse HAKAI showed significantly increased cell scattering and increased E-cadherin endocytosis after addition of HGF. Thus, in mice, HAKAI appears to control E-cadherin levels at the plasma membrane.

Identification of a human homolog of HAKAI would provide reagents and methods for treating proliferative disorders, including cancer.

### BRIEF SUMMARY OF THE INVENTION

The invention provides at least the following embodiments.

One embodiment of the invention is a method of decreasing hsHAKAI activity in a cell. An expression product of an hsHAKAI gene is contacted with a reagent that specifically binds to the expression product. The hsHAKAI activity is thereby decreased in the cell.

Another embodiment of the invention is a method of screening for candidate therapeutic agents for treating proliferative disorders. A protein comprising the amino acid sequence shown in SEQ ID NO:2 is contacted with a test compound. Binding between the protein and test compound is assayed. A test compound that binds to the protein is identified as a potential therapeutic agent for treating proliferative disorders.

Yet another embodiment of the invention is a method of screening for candidate therapeutic agents for treating proliferative disorders. Expression of a polynucleotide comprising the nucleotide sequence shown in SEQ ID NO:1 is assayed in the presence and absence of a test compound. A test compound that decreases expression is identified as a candidate therapeutic agent for treating proliferative disorders.

Even another embodiment of the invention is a method of screening for candidate therapeutic agents for treating proliferative disorders. A first protein, a second protein, and a test compound are contacted. The first protein comprises hsHAKAI and the second protein comprises E-cadherin or the first protein comprises E-cadherin and the second protein comprises hsHAKAI. The quantity of the first protein which is bound to, is displaced from, or is prevented from binding to, the second protein is determined. A test compound that decreases the quantity of the first protein bound to the second protein, or which displaces the first protein bound to the second protein, or which prevents the first protein from binding to the second protein, is identified as a candidate therapeutic agent for treating proliferative disorders.

Even another embodiment of the invention is a method of screening for candidate therapeutic agents for treating proliferative disorders. A test compound to be tested is contacted with a yeast cell comprising (1) two fused gene constructs, wherein a first construct comprises a yeast GAL-4 binding domain and a coding sequence selected from the group consisting of a coding sequence for hsHAKAI and a coding sequence for E-cadherin, and wherein a second construct comprises a yeast GAL-4 activation domain and a domain selected from the group consisting of: a coding sequence for hsHAKAI and a coding sequence for E-cadherin, wherein when the first construct comprises a coding sequence for E-cadherin, the second construct comprises a coding sequence for hsHAKAI, and when the second construct comprises a coding sequence for hsHAKAI, the first construct comprises a coding sequence for E-cadherin; and (2) a β-galactosidase reporter gene under the control of a yeast GAL-4 promoter, which is activated by the gene products of the two fused gene constructs. Expression of β-galactosidase in the yeast cell is detected. A test compound that decreases expression of β-galactosidase relative to expression of β-galactosidase in the absence of the test compound is identified as a candidate therapeutic agent for treating proliferative disorders.

A further embodiment of the invention is a yeast cell comprising (1) two fused gene constructs, wherein a first construct comprises a yeast GAL-4 binding domain and a coding sequence selected from the group consisting of a coding sequence for hsHAKAI and a coding sequence for E-cadherin, and wherein a second construct comprises a yeast GAL-4 activation domain and a domain selected from the group consisting of: a coding sequence for hsHAKAI and a coding sequence for E-cadherin, wherein when the first construct comprises a coding sequence for E-cadherin, the second construct comprises a coding sequence for hsHAKAI, and when the second construct comprises a coding sequence for hsHAKAI, the first construct comprises a coding sequence for E-cadherin; and (2) a β-galactosidase reporter gene under the control of a yeast GAL-4 promoter, which is activated by the gene products of the two fused gene constructs.

Still another embodiment of the invention is a pharmaceutical composition comprising a reagent that specifically binds to a polynucleotide encoding hsHAKAI comprising the amino acid sequence shown in SEQ ID NO:2 and a pharmaceutically acceptable carrier.

Another embodiment of the invention is a pharmaceutical composition comprising a reagent that specifically binds to a protein comprising the amino acid sequence shown in SEQ ID NO:2 and a pharmaceutically acceptable carrier.

### BRIEF DESCRIPTION OF THE FIGURES

FIG. 1. Time course of hsHAKAI expression in SW620 cells treated with antisense oligonucleotide C245-1 (SEQ ID NO:3).

FIG. 2. Depletion of hsHAKAI mRNA in MDA435 cells after transfection with interference RNA C245 (SEQ ID NO:5).

FIG. 3. Inhibition of proliferation of SW620 cells treated with antisense oligonucleotide C245-1 (SEQ ID NO:3).

FIG. 4. Inhibition of anchorage-independent growth of SW620 cells after transfection with C245-1 antisense-oligonucleotide (SEQ ID NO:3).

FIG. 5. Inhibition of proliferation of MDA-MB-435 cells treated with antisense oligonucleotide C245-1 (SEQ ID NO:3).

### DETAILED DESCRIPTION OF THE INVENTION

A human homolog of the mouse HAKAI gene, identified with GenBank Accession No. NM_{—}024814, LocusLink ID 79872, was identified by BLAST searching against the GenBank cDNA database. The coding of NM_{—}024814 is shown in SEQ ID NO:1; the amino acid sequence of human HAKAI protein (“hsHAKAI”) is shown in SEQ ID NO:2. The human and mouse coding sequences are 93% identical over 1425 base pairs.

Reagents that decrease effective levels of hsHAKAI (e.g., by inhibiting hsHAKAI gene expression, inhibiting binding to hsHAKAI and E-cadherin, or inhibiting hsHAKAI enzymatic activity) can be used to treat cancer and other proliferative disorders, such as such as dysplasias and hyperplasias. Neoplasias which can be treated include, but are not limited to, melanomas, squamous cell carcinomas, adenocarcinomas, hepatocellular carcinomas, renal cell carcinomas, sarcomas, myosarcomas, non-small cell lung carcinomas, leukemias, lymphomas, osteosarcomas, central nervous system tumors such as gliomas, astrocytomas, oligodendrogliomas, and neuroblastomas, tumors of mixed origin, such as Wilms' tumor and teratocarcinomas, and metastatic tumors. Proliferative disorders that can be treated include disorders such as anhydric hereditary ectodermal dysplasia, congenital alveolar dysplasia, epithelial dysplasia of the cervix, fibrous dysplasia of bone, and mammary dysplasia. Hyperplasias, for example, endometrial, adrenal, breast, prostate, or thyroid hyperplasias, or pseudoepitheliomatous hyperplasia of the skin, also can be treated.

Inhibition of hsHAKAI Gene Expression

One aspect of the invention involves inhibiting the level of hsHAKAI gene expression. Preferably, the reagent used to inhibit the level of hsHAKAI gene expression decreases the level of gene expression by at least 50%, 60%, 70%, or 80%. Most preferably, the level of gene expression is decreased by at least 90%, 95%, 99%, or 100%. The effectiveness of the mechanism chosen to inhibit hsHAKAI gene expression can be assessed using methods well known in the art, such as hybridization of nucleotide probes to hsHAKAI mRNA, quantitative RT-PCR, or detection of hsHAKAI protein using specific antibodies.

Antisense Oligonucleotides

In one embodiment of the invention, hsHAKAI gene expression is inhibited using an antisense oligonucleotide. The nucleotide sequence of the antisense oligonucleotide is complementary to at least a portion of the sequence encoding hsHAKAI, which can be selected from the nucleotide sequence shown in SEQ ID NO:1. Preferably, the antisense oligonucleotide sequence is at least 11 nucleotides in length, but can be at least 12, 15, 20, 25, 30, 35, 40, 45, or 50 or more nucleotides long. Longer sequences can also be used. An example of an hsHAKAI antisense oligonucleotide is shown in SEQ ID NO:3.

Antisense oligonucleotides can be deoxyribonucleotides, ribonucleotides, or a combination of both. Oligonucleotides can be synthesized manually or by an automated synthesizer, by covalently linking the 5′ end of one nucleotide with the 3′ end of another nucleotide with non-phosphodiester internucleotide linkages such alkylphosphonates, phosphorothioates, phosphorodithioates, alkylphosphonothioates, alkylphosphonates, phosphoramidates, phosphate esters, carbamates, acetamidate, carboxymethyl esters, carbonates, and phosphate triesters. See Brown, Meth. Mol. Biol. 20:1-8, 1994; Sonveaux, Meth. Mol. Biol. 26:1-72, 1994; Uhlmann et al., Chem. Rev. 90:543-583, 1990.

Although precise complementarity is not required for successful duplex formation between an antisense molecule and the complementary coding sequence of an hsHAKAI gene, antisense molecules with no more than one mismatch are preferred. One skilled in the art can easily use the calculated melting point of an antisense-sense pair to determine the degree of mismatching which will be tolerated between a particular antisense oligonucleotide and a particular coding sequence.

Antisense oligonucleotides can be modified without affecting their ability to hybridize to an hsHAKAI coding sequence. These modifications can be internal or at one or both ends of the antisense molecule. For example, internucleoside phosphate linkages can be modified by adding cholesteryl or diamine moieties with varying numbers of carbon residues between the amino groups and terminal ribose. Modified bases and/or sugars, such as arabinose instead of ribose, or a 3′,5′-substituted oligonucleotide in which the 3′ hydroxyl group or the 5′ phosphate group are substituted, can also be employed in a modified antisense oligonucleotide. These modified oligonucleotides can be prepared by methods well known in the art. See, e.g., Agrawal et al., Trends Biotechnol. 10:152-158, 1992; Uhlmann et al., Chem. Rev. 90:543-584, 1990; Uhlmann et al., Tetrahedron. Lett. 215:3539-3542, 1987.

Antisense oligonucleotides can be transferred to a cell by any method known in the art. For example, cells can be transfected with an expression construct capable of generating the antisense oligonucleotide as a transcription product, e.g., by including the antisense oligonucleotide in a viral vector, such as a retroviral vector, adenoviral vector, or the like. See U.S. Pat. Nos. 5,922,857 and 4,593,002 and Mukhopadhyay et al., Cancer Research 51, 1744-48, 1991. Mechanical methods, such as microinjection, liposome-mediated transfection, electroporation, or calcium phosphate precipitation, can be used to introduce the construct into cells in which it is desired to decrease hsHAKAI expression.

Alternatively, if it is desired that the cells stably retain the construct, it can be supplied on a plasmid and maintained as a separate element or integrated into the genome of the cells, as is known in the art. The construct can include transcriptional regulatory elements, such as a promoter element, an enhancer or UAS element, and a transcriptional terminator signal, for controlling transcription of the antisense oligonucleotide in the transfected cells.

Alternatively, an antisense oligonucleotide can be administered to a cell in a vehicle such as a liposome or a lipid suspension such as N-[(1-(2,3-dioleoyloxy)propyl]-N,N,N-trimethylammonium methylsulfate (DOTAP), N-[1-(2,3-dioleoyloxy)propyl]-N,N,N-trimethylammonium chloride (DOTMA), and the like. An antisense oligonucleotide also can be linked to a moiety that increases cellular uptake of the oligonucleotide. This moiety may be hydrophobic, such as a phospholipid or a lipid such as a steroid (e.g., cholesterol), or may be polycationic (e.g., polylysine). The hydrophobic or polycationic moiety is attached at any point to the antisense oligonucleotide, including at the 3′ or 5′ end, base, sugar hydroxyls, and internucleoside linkages.

A particularly preferred moiety to increase uptake is a cholesteryl group. Cholesteryl-like groups may be attached through an activated cholesteryl chloroformate, for example, or cholic acid. See Letsinger et al., Proc. Natl. Acad. Sci. USA 86, 6553-56, 1989.

Ribozymes

In another embodiment of the invention, a ribozyme (i.e., an RNA molecule with catalytic activity), is used to decrease hsHAKAI levels. See, e.g., Cech, Science 236, 1532-39, 1987; Cech, Ann. Rev. Biochem. 59, 543-68, 1990, Cech, Curr. Opin. Struct. Biol. 2: 605-09, 1992; Couture & Stinchcomb, Trends Genet. 12, 510-15, 1996. Ribozymes can be used to inhibit gene function by cleaving an RNA sequence, as is known in the art (e.g., Haseloff et al., U.S. Pat. No. 5,641,673). Ribozymes can be introduced into cells by the same methods used for administration of antisense oligonucleotides described above.

An hsHAKAI coding sequence can be used to generate ribozymes that will specifically bind to mRNA transcribed from the hsHAKAI gene. Methods of designing and constructing ribozymes which can cleave other RNA molecules in trans in a highly sequence specific manner have been developed and described in the art (see Haseloff et al., Nature 334, 585-91, 1988). For example, the cleavage activity of ribozymes can be targeted to specific RNAs by engineering a discrete “hybridization” region into the ribozyme. The hybridization region contains a sequence complementary to the target RNA and thus specifically hybridizes with the target (see, for example, Gerlach et al., EP 321,201). The coding sequence shown in SEQ ID NO:1 provides a source of suitable hybridization region sequences. Longer complementary sequences can be used to increase the affinity of the hybridization sequence for the target. The hybridizing and cleavage regions of the ribozyme can be integrally related; thus, upon hybridizing to the target RNA through the complementary regions, the catalytic region of the ribozyme can cleave the target.

As taught in Haseloff et al., U.S. Pat. No. 5,641,673, ribozymes can be engineered so that ribozyme expression will occur in response to factors that induce expression of a target gene. Ribozymes can also be engineered to provide an additional level of regulation, so that destruction of mRNA occurs only when both a ribozyme and a target gene are induced in the cells.

Interference RNA

hsHAKAI expression also can be lowered by degrading hsHAKAI mRNA using an interference RNA, i.e., a double-stranded RNA that results in catalytic degradation of mRNA. Methods of using of interference RNA to lower gene expression are known in the art. Any of these methods can be used to inhibit hsHAKAI gene expression. See Fire et al., Nature 391, 806-11, 1998; Fire, Trends Genet. 15, 358-63, 1999; Sharp, RNA interference 2001,” Genes Dev. 15, 485-90, 2001; Hammond et al., Nature Rev. Genet. 2, 110-19, 2001; Tuschl, Chem. Biochem. 2, 239-45, 2001; Hamilton et al., Science 286, 950-52, 1999; Hammond et al., Nature 404, 293-96, 2000; Zamore et al., Cell 101, 25-33, 2000; Bernstein et al., Nature 409, 363-66, 2001; Elbashir et al., Genes Dev. 15, 188-200, 2001; WO 01/29058; WO 99/32619; Elbashir et al., Nature 411, 494-98, 2001; US 2002/0022029.

Decreasing Effective Levels of hsHAKAI Protein

Effective levels of hsHAKAI protein can be decreased, for example, by inhibiting the E3-ubiquitin ligase activity of hsHAKAI or by disrupting binding between hsHAKAI and E-cadherin.

Antibodies

Antibodies can be used to decrease effective levels of hsHAKAI, for example by preventing binding between hsHAKAI and E-cadherin or by blocking enzymatic activity of hsHAKAI. To prevent hsHAKAI-E-cadherin binding, either an antibody that specifically binds to hsHAKAI or one that specifically binds to E-cadherin can be used. To inhibit hsHAKAI enzymatic activity, an antibody preferably binds to the active site of hsHAKAI or binds to otherwise blocks the active site such that normal levels of enzymatic activity are decreased.

Any type of antibody known in the art can be generated to bind specifically to an epitope of hsHAKAI or E-cadherin. “Antibody” as used herein includes intact immunoglobulin molecules, as well as fragments thereof, such as Fab, F(ab′)₂, and Fv, which are capable of binding an epitope of hsHAKAI or E-cadherin. Typically, at least 6, 8, 10, or 12 contiguous amino acids are required to form an epitope. However, epitopes which involve non-contiguous amino acids may require more, e.g., at least 15, 25, or 50 amino acids.

Monoclonal and other antibodies also can be “humanized” to prevent a patient from mounting an immune response against the antibody when it is used therapeutically. Such antibodies may be sufficiently similar in sequence to human antibodies to be used directly in therapy or may require alteration of a few key residues. Sequence differences between rodent antibodies and human sequences can be minimized by replacing residues which differ from those in the human sequences by site directed mutagenesis of individual residues or by grating of entire complementarity determining regions. Alternatively, humanized antibodies can be produced using recombinant methods, as described in GB2188638B. Antibodies that specifically bind to hsHAKAI or to E-cadherin can contain antigen binding sites which are either partially or fully humanized, as disclosed in U.S. Pat. No. 5,565,332.

An antibody that specifically binds to an epitope of hsHAKAI or E-cadherin can be used therapeutically, as well as in immunochemical assays, such as Western blots, ELISAs, radioimmunoassays, immunohistochemical assays, immunoprecipitations, or other immunochemical assays known in the art. Various immunoassays can be used to identify antibodies having the desired specificity. Numerous protocols for competitive binding or immunoradiometric assays are well known in the art. Such immunoassays typically involve the measurement of complex formation between an immunogen and an antibody that specifically binds to the immunogen.

Typically, an antibody that specifically binds to hsHAKAI or E-cadherin provides a detection signal at least 5-, 10-, or 20-fold higher than a detection signal provided with other proteins when used in an immunochemical assay. Preferably, antibodies that specifically bind to hsHAKAI or E-cadherin do not detect other proteins in immunochemical assays and can immunoprecipitate hsHAKAI or E-cadherin from solution.

Polynucleotides encoding single-chain antibodies of the invention can be introduced into cells as described above. Antibodies themselves can be administered in pharmaceutical compositions of the invention, as described below.

Screening for Candidate Therapeutic Agents

The invention provides methods of screening test compounds for candidate therapeutic agents that can be used to treat proliferative disorders by inhibiting the activity of hsHAKAI or by blocking its binding to E-cadherin. A test compound preferably decreases hsHAKAI's E3 ubiquitin ligase activity or binding to E-cadherin by at least about 10, preferably about 50, more preferably about 75, 90, or 100% relative to the absence of the test compound.

Test Compounds

Test compounds can be pharmacologic agents already known in the art or can be compounds previously unknown to have any pharmacological activity. The compounds can be naturally occurring or designed in the laboratory. They can be isolated from microorganisms, animals, or plants, and can be produced recombinantly, or synthesized by chemical methods known in the art. If desired, test compounds can be obtained using any of the numerous combinatorial library methods known in the art, including but not limited to, biological libraries, spatially addressable parallel solid phase or solution phase libraries, synthetic library methods requiring deconvolution, the “one-bead one-compound” library method, and synthetic library methods using affinity chromatography selection. Methods for the synthesis of molecular libraries are well known in the art.

High Throughput Screening

Test compounds can be screened for the ability to disrupt hsHAKAI-E-cadherin binding or to inhibit hsHAKAI's E3 ubiquitin ligase activity using high throughput screening so that many discrete compounds can be tested quickly and in parallel. The most widely established techniques utilize 96-well microtiter plates. The wells of the microtiter plates typically require assay volumes that range from 50 to 500 μl. In addition to the plates, many instruments, materials, pipettors, robotics, plate washers, and plate readers are commercially available to fit the 96-well format. Alternatively, “free format” assays can be used. See, e.g., Jayawickreme et al., Proc. Natl. Acad. Sci. U.S.A. 19, 1614-18, 1994, Salmon et al., Molecular Diversity 2, 57-63, 1996, and U.S. Pat. No. 5,976,813.

Binding Assays

Any binding assays known in the art can be used to identify test compounds that bind to hsHAKAI or E-cadherin or that disrupt the binding between hsHAKAI and E-cadherin. In some binding assays, either the test compound or the test protein (either hsHAKAI or E-cadherin or a fusion protein comprising either hsHAKAI or E-cadherin) can comprise a detectable label, such as a fluorescent, radioisotopic, chemiluminescent, or enzymatic label (e.g., horseradish peroxidase, alkaline phosphatase, or luciferase). Binding between a test compound and the test protein can be detected, for example, by direct counting of radioemmission, by scintillation counting, or by determining conversion of an appropriate substrate to a detectable product. Alternatively, binding between a test compound and the test protein can be determined without labeling either of the interactants. For example, a microphysiometer (e.g., Cytosensor™) can be used to detect binding of a test compound with hsHAKAI. See McConnell et al., Science 257, 1906-12, 1992. Real-time Bimolecular Interaction Analysis (BIA) also can be used, as described in Sjolander & Urbaniczky, Anal. Chem. 63, 2338-45, 1991, and Szabo et al., Curr. Opin. Struct. Biol. 5, 699-705, 1995.

In yet another aspect of the invention, either hsHAKAI or E-cadherin can be used as a “bait protein” in a two-hybrid assay or three-hybrid assay employing a yeast cell comprising constructs encoding. See, e.g., U.S. Pat. No. 5,283,317; Zervos et al., Cell 72, 223-232, 1993; Madura et al., J. Biol. Chem. 268, 12046-12054, 1993; Bartel et al., BioTechniques 14, 920-924, 1993; Iwabuchi et al., Oncogene 8, 1693-1696, 1993; and Brent WO94/10300. Such assays typically employ a yeast cell comprising two fused gene constructs and a reporter gene (e.g., β-galactosidase) under the control of a yeast GAL-4 promoter. One of the fused gene constructs comprises a yeast GAL-4 binding domain and a coding sequence for either hsHAKAI or E-cadherin. Coding sequences for human E-cadherin are known in the art. The second fused gene construct comprises one of the coding sequences and a yeast GAL-4 activation domain. If the first construct comprises a coding sequence for E-cadherin, the second construct comprises a coding sequence for hsHAKAI, and vice versa. The reporter gene is activated by the gene products of the two fused gene constructs. Expression of the reporter gene in the cell is detected, and test compounds that decrease expression of the reporter gene relative to its expression in the absence of the test compounds are identified as candidate therapeutic agents for treating proliferative disorders.

Either the test compound or the test protein can be immobilized to facilitate separation of bound from unbound forms of one or both of the interactants, as well as to accommodate automation of the assay. Thus, either the test protein or the test compound can be bound to a solid support. Suitable solid supports include, but are not limited to, glass or plastic slides, tissue culture plates, microtiter wells, tubes, silicon chips, or particles such as beads (including, but not limited to, latex, polystyrene, or glass beads). Any method known in the art can be used to attach the test protein or the test compound to a solid support, including use of covalent and non-covalent linkages, passive absorption, or pairs of binding moieties attached respectively to the test protein or the test compound and the solid support. Test compounds preferably are bound to the solid support in an array, so that the location of individual test compounds can be tracked. Binding of a test compound to the test protein can be accomplished in any vessel suitable for containing the reactants. Examples of such vessels include microtiter plates, test tubes, and microcentrifuge tubes.

Screening for test compounds that bind to hsHAKAI also can be carried out in an intact cell. Any cell which comprises hsHAKAI can be used in a cell-based assay system. The hsHAKAI can be naturally occurring in the cell or can be introduced using techniques such as those described above. Test compounds able to enter the cell are tested for binding to hsHAKAI as described above.

Enzymatic Activity

Test compounds can be tested for the ability to inhibit the enzymatic activity of hsHAKAI. E3 ubiquitin ligase activity of hsHAKAI can be measured, for example, as described in Hatakeyama, et al., J. Biol. Chem. 272, 15085, 1997, or U.S. Pat. No. 6,087,122. Enzyme assays can be carried out after contacting either purified hsHAKAI or an intact cell with a test compound. A test compound that decreases enzymatic activity of hsHAKAI by at least about 10, preferably about 50, more preferably about 75, 90, or 100% is identified as a potential therapeutic agent for treating proliferative disorders.

Pharmaceutical Compositions

Compositions comprising reagents that decrease effective levels of hsHAKAI can optionally comprise a pharmaceutically acceptable carrier. Pharmaceutically acceptable carriers are well known to those in the art. Such carriers include, but are not limited to, large, slowly metabolized macromolecules, such as proteins, polysaccharides, polylactic acids, polyglycolic acids, polymeric amino acids, amino acid copolymers, and inactive virus particles. Pharmaceutically acceptable salts can also be used in compositions of the invention, for example, mineral salts such as hydrochlorides, hydrobromides, phosphates, or sulfates, as well as salts of organic acids such as acetates, proprionates, malonates, or benzoates. Pharmaceutical compositions can also contain liquids, such as water, saline, glycerol, and ethanol, as well as substances such as wetting agents, emulsifying agents, or pH buffering agents. Liposomes, such as those described in U.S. Pat. No. 5,422,120, WO 95/13796, WO 91/14445, or EP 5 249 68 B1, can also be used as a carrier for a pharmaceutical composition of the invention.

Typically, a pharmaceutical composition of the invention is prepared as an injectable, either as a liquid solution or suspension; however, solid forms suitable for solution or suspension in liquid vehicles prior to injection can also be prepared. A pharmaceutical composition of the invention can also be formulated into an enteric-coated tablet or gel capsule according to known methods in the art, such as those described in U.S. Pat. No. 4,853,230, EP 2 251 89, AU 9,224,296, and AU 9,230,801.

Therapeutic Administration

A pharmaceutical composition comprising all or a portion of a reagent that decreases effective levels of hsHAKAI can be administered to treat proliferative disorders. Various methods can be used to administer the composition directly to a specific site in the body. For treatment of a tumor, for example, a pharmaceutical composition can be injected several times in several different locations within the body of the tumor. Alternatively, arteries that serve the tumor can be identified, and a pharmaceutical composition can be injected into such an artery in order to deliver the composition to the tumor.

A tumor that has a necrotic center can be aspirated, and a pharmaceutical composition of the invention can be injected directly into the now empty center of the tumor. Alternatively, a pharmaceutical composition also can be administered directly to the surface of a tumor, for example, by topical application of the composition. X-ray imaging can be used to assist in certain of these delivery methods. If desired, pharmaceutical compositions of the invention can be administered simultaneously or sequentially together with other therapeutic agents.

Pharmaceutical compositions of the invention can be delivered to specific tissues using receptor-mediated targeted delivery. Receptor-mediated DNA delivery techniques are taught in, for example, Findeis et al. Trends in Biotechnol. 11, 202-05, (1993); Chiou et al., GENE THERAPEUTICS: METHODS AND APPLICATIONS OF DIRECT GENE TRANSFER (J. A. Wolff, ed.) (1994); Wu & Wu, J. Biol. Chem. 263, 621-24, 1988; Wu et al., J. Biol. Chem. 269, 542-46, 1994; Zenke et al., Proc. Natl. Acad. Sci. U.S.A. 87, 3655-59, 1990; Wu et al., J. Biol. Chem. 266, 338-42, 1991.

Both the dose of a particular pharmaceutical composition and the means of administering the composition can be determined based on specific qualities of the composition, the condition, age, and weight of the patient, the progression of the particular disease being treated, and other relevant factors. If the composition contains antibodies, effective dosages of the composition typically are in the range of about 5 μg to about 50 μg/kg of patient body weight, about 50 μg to about 5 mg/kg, about 100 μg to about 500 μg/kg of patient body weight, and about 200 to about 250 μg/kg. Compositions containing, for example, antisense oligonucleotides, ribozymes, iRNA, or single chain antibody-encoding sequences, can be administered in a range of about 100 ng to about 200 mg of DNA for local administration. Suitable concentrations range from about 500 ng to about 50 mg, about 1 μg to about 2 mg, about 5 μg to about 500 μg, and about 20 μg to about 100 μg of DNA. Factors such as method of action and efficacy of transformation and expression are considerations that will affect the dosage required for ultimate efficacy of the pharmaceutical composition. In all cases, routine experimentation in clinical trials will determine specific ranges for optimal therapeutic effect.

All patents, patent applications, and references cited in this disclosure are expressly incorporated herein by reference in their entireties. The above disclosure generally describes the present invention. A more complete understanding can be obtained by reference to the following specific examples, which are provided for purposes of illustration only and are not intended to limit the scope of the invention.

### EXAMPLE 1

Transfection of Mammalian Cells with Antisense Oligonucleotides or Interference RNA

SW620, MDA435, or SW620 cells were plated at 70-80% confluency. For transfection with antisense oligonucleotides, cells were incubated in transfection mixture containing 300 nM antisense or reverse control oligonucleotides with lipidoid carrier (ratio 1:3) for at least four hours.

For transfection with interference RNA (siRNA), cells were incubated in transfection mixture containing 100 nM siRNA for at least four hours. HAKAI/C245 siRNA nucleotide sequence (AAGCTCATCTCCAAACAAGCA, SEQ ID NO:5) was designed using NM_{—}024814 (SEQ ID NO:1) as template and purchased from Dharmacon Research, Lafayette, Colo.

### EXAMPLE 2

Effect of hsHAKAI on mRNA Levels

Total RNA was extracted from transfected cells using the High Pure RNA Isolation Kit from Roche following the protocol provided by the manufacturer. Following extraction, the RNA was reverse-transcribed for use as a PCR template. Generally 0.2-1 μg of total RNA was added to a buffer/enzyme mixture containing Reverse Transcriptase (Ambion, Inc.) and incubated for 1 hour at 42° C.

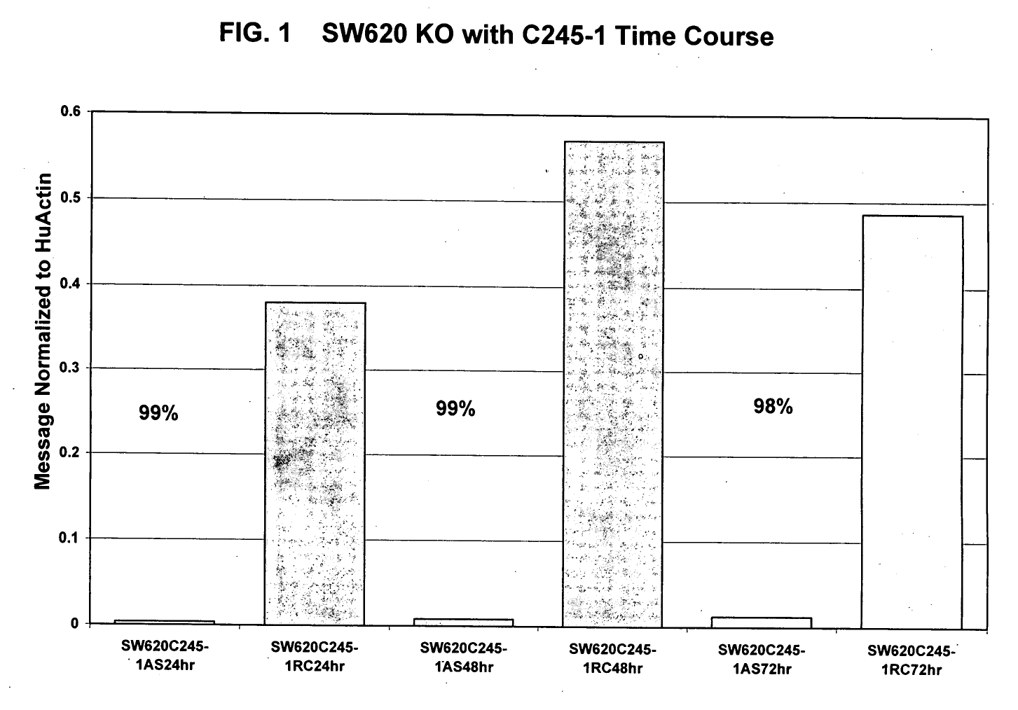

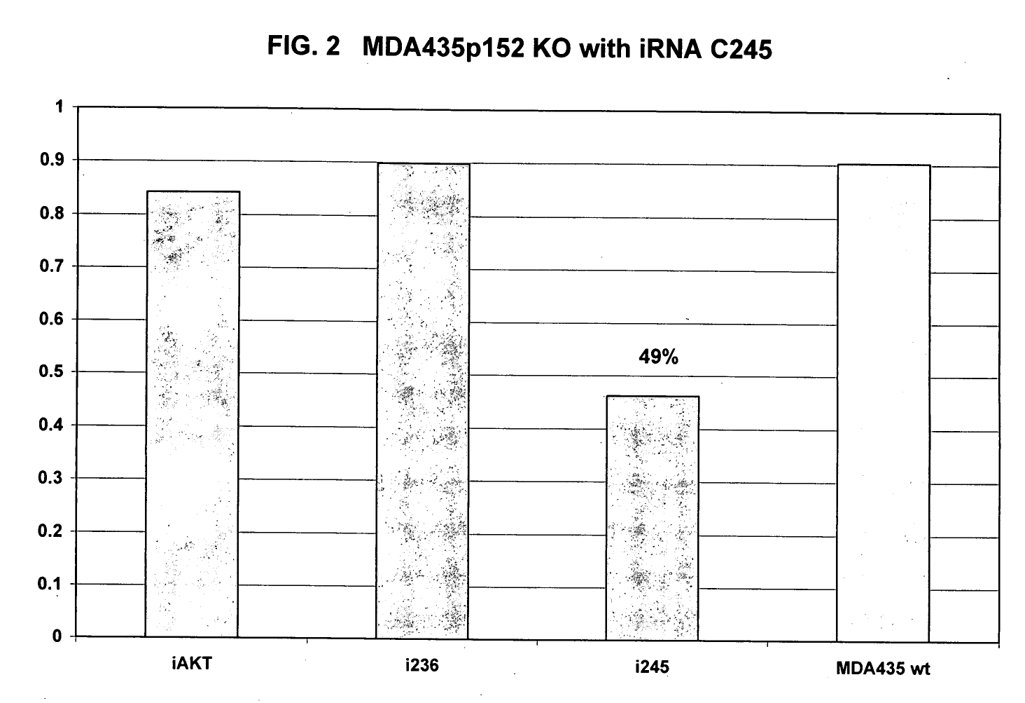

Following reverse transcription, target genes were amplified using the Applied Biosystems 5700 or 7000 Sequence Detection System, which is a real-time PCR machine. The amount of PCR product was detected using SYBR Green (Molecular Probes, Eugene, Oreg.), a dye that fluoresces after binding to double stranded DNA. Amounts of amplified target sequences obtained from each PCR reaction were normalized through comparison with an internal control (e.g., beta-actin). FIGS. 1 and 2 show the relative levels of HAKAI mRNA in cells, normalized to actin. If not stated differently, cells were harvested 24 hours after transfection. Wt=untransfected cells.

### EXAMPLE 3

Effect of hsHAKAI on Cell Proliferation

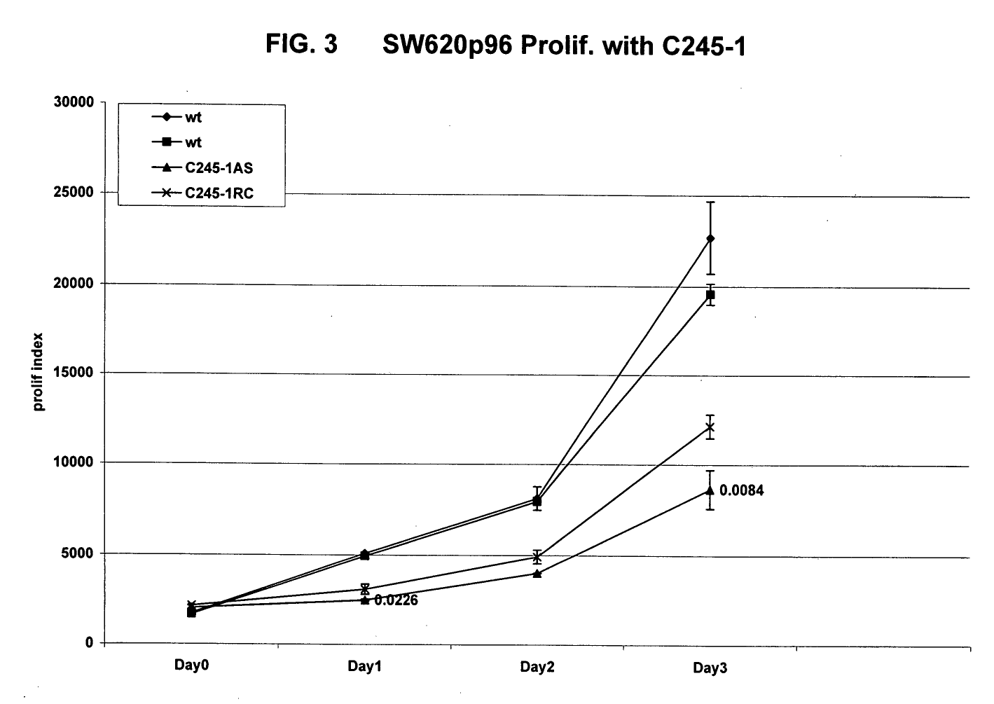

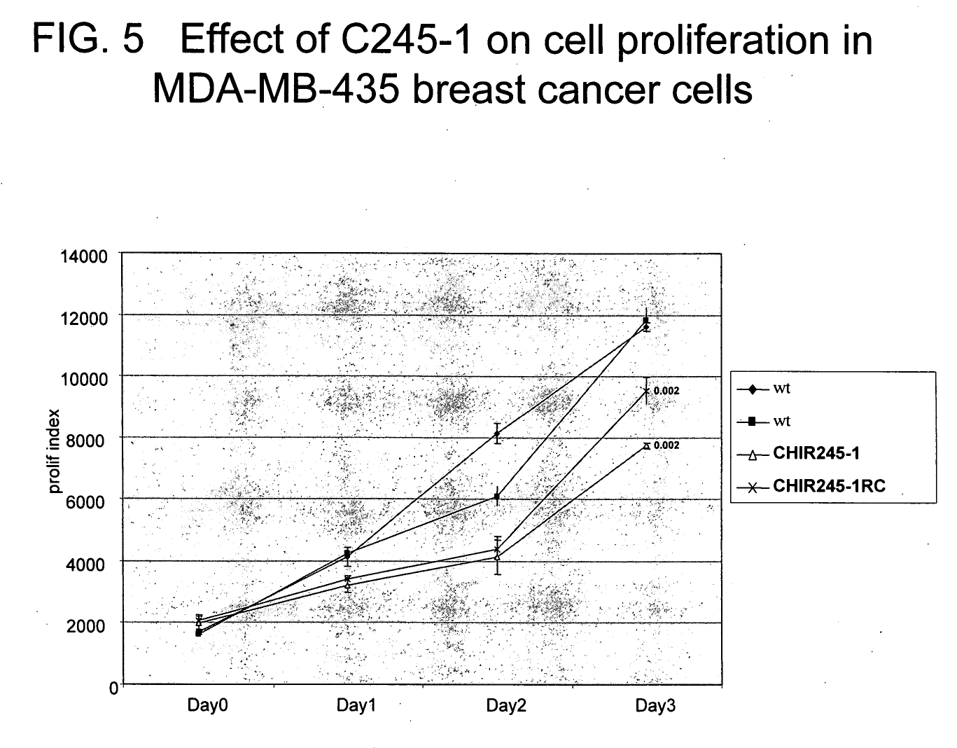

To demonstrate that hsHAKAI is required for cell proliferation, we performed a CellTiter-Glo Luminescent Cell Viability Assay (Promega). We transfected SW620 and MDA-MB-435 cells with antisense (SEQ ID NO:3) or reverse control (SEQ ID NO:4) oligonucleotides. One hundred microliters of the transfection mixture containing 10,000 cells was plated per well on a 96-well plate. Each transfection was plated in triplicate, and a total of four plates were tested. One plate was harvested each day, beginning with the day of transfection. To detect viable cells, the amount of ATP present was quantitated by adding 100 μl of CellTiter-Glo reagent and reading the plate in a luminometer. The results are shown in FIGS. 3 and 5. The difference between C245-1AS and C245-1RC transfected cells is significant as indicated by a p-value<0.05.

### EXAMPLE 4

Effect of hsHAKAI on Anchorage-Independent Growth in Tumor Cells

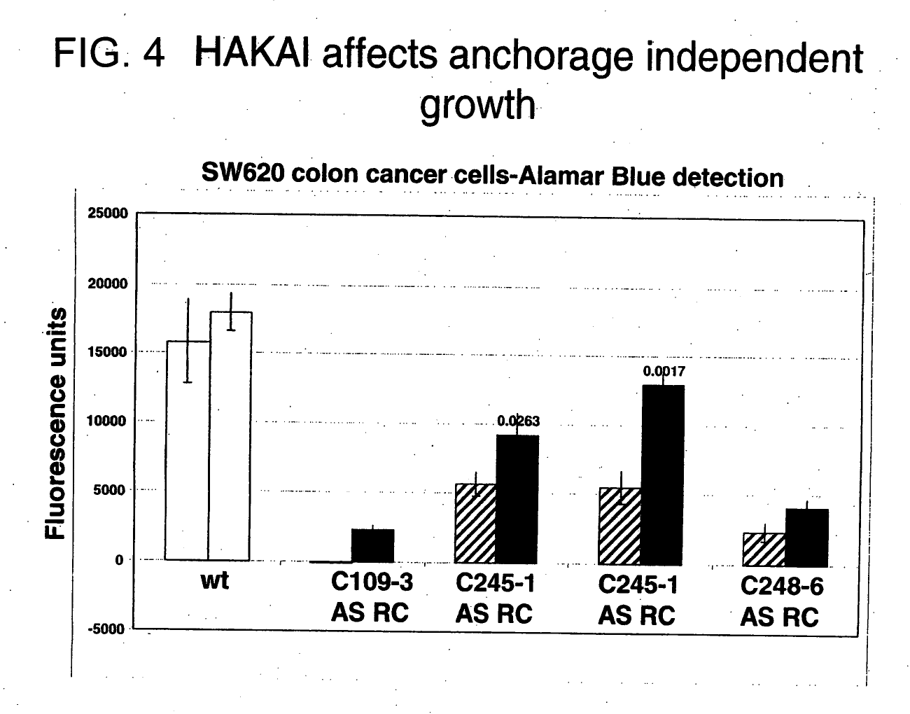

To demonstrate the effect of hsHAKAI on anchorage independent growth in tumor cells, we performed a 96-well soft agarose assay. First, the 96-well plate was treated with polyHEME (Sigma) to prevent attachment of cells to the plastic. SW620 cells were transfected as described in Example 1 with antisense (SEQ ID NO:3) or with reverse control (SEQ ID NO:4) oligonucleotides. The next day, the transfected cells were harvested and plated at a concentration 500 cells/well in 150 μl medium containing 0.3% of melted Agarose (v/v). Each transfection was plated in triplicate. Ten minutes later, 100 μl of medium was added on top of the solidified agarose layer. The plates were incubated at 37° C. for one week. The number of viable cells was determined by adding 25 μl of Alamar Blue (Trek Diagnostics) and determining fluorescence at OD₅₉₀ at various time points. Colonies also could be counted using a microscope. The results are shown in FIG. 4.

1. A method of decreasing hsHAKAI activity in a cell, comprising the step of:
contacting the cell with a nucleic acid reagent that specifically binds to a polynucleotide encoding hsHAKAI, thereby decreasing hsHAKAI activity in the cell.

2. The method of claim 1 wherein the polynucleotide is mRNA.

3. The method of claim 1 wherein the nucleic acid reagent is an antisense oligonucleotide.

4. The method of claim 3 wherein the antisense oligonucleotide comprises the nucleotide sequence SEQ ID NO:3.

5. The method of claim 1 wherein the nucleic acid reagent is an siRNA.

6. The method of claim 5 wherein the siRNA comprises the nucleotide sequence SEQ ID NO:5.

7. The method of claim 1 wherein the nucleic acid reagent is an interference RNA.

8. The method of claim 1 wherein the cell is in vitro.

9. The method of claim 1 wherein the cell is in vivo.

10. The method of claim 1 wherein the hsHAKAI comprises the amino acid sequence SEQ ID NO:2.

11. A composition, comprising:
a nucleic acid reagent that specifically binds to a polynucleotide encoding hsHAKAI; and
a pharmaceutically acceptable carrier.

12. The composition of claim 11 wherein the nucleic acid reagent is an antisense oligonucleotide.

13. The composition of claim 12 wherein the antisense oligonucleotide comprises the nucleotide sequence SEQ ID NO:3.

14. The composition of claim 11 wherein the nucleic acid reagent is an siRNA.

15. The composition of claim 14 wherein the siRNA comprises the nucleotide sequence SEQ ID NO:5.

16. The composition of claim 11 wherein the nucleic acid reagent is an interference RNA.


SEQUENCE LISTING (5 sequences)

SEQ ID NO: 1 - DNA, 1476 bp, Homo sapiens
atggatcacactgacaatgagttacaaggcactaatagttctggatccttgggtggtcttgatgttcgcagacgaattcctataaagctcatctccaaacaagcaaacaaagcgaaacctgcaccgcgaactcaaagaactataaacaggatgcctgcaaaggctccacctggtgatgaagaaggatttgattataatgaagaagaacggtatgactgtaaagggggtgagctgtttgcaaatcagcgaagatttcctggacaccttttttgggactttcagataaacatcttaggtgaaaaggatgatacaccagttcatttctgtgacaagtgtggattgcctattaaaatctatgggagaatgattccatgcaagcatgttttttgctatgactgtgctattttacatgaaaaaaagggagataagatgtgtccaggctgtagtgatcctgtgcagcgaattgagcagtgtacacgaggttctctcttcatgtgtagcattgttcaagggtgcaagagaacatatttgtctcagagagacttacaggctcatatcaaccatcgccatatgagagctggaaaacctgttacccgtgcttcacttgaaaatgttcatcctcctattgccccaccaccaactgaaatccctgagcgttttataatgccaccagacaagcaccatatgagccatattccgccaaagcagcatatcatgatgccaccacctcctttgcaacatgtgccacatgagcactataatcagccacatgaggatattcgtgctcctccagcagaattgtccatggctccacctccacctcgatcggtcagtcaggaaacctttcgtatttcaacaagaaaacacagcaatttaataaccgtccctattcaggatgactcaaattcaggtgctagagaaccaccacctcctgccccagcacctgctcaccatcatcctgaatatcagggtcaaccagtggtatcgcaccctcatcatattatgcctccacagcaacattatgcaccacccccacctcctccaccaccaataagccatccaatgccacatcctccccaggctgcaggtactcctcacttggtatatagccaagctccacctccaccaatgacctctgctccaccaccaataacccctccccctggacatattattgcccagatgccaccttatatgaatcatcctcctccaggacctcccccgcctcaacatggtggtccacctgtaactgcaccccctcctcaccattataatcctaactcattaccccagttcactgaagatcaaggaactccgagccctccatttacacaaccagggggaatgagtcctggtatatggcctgcaccaagagggccaccaccacctccacgattgcagggtccgccttctcaaaccccacttcctggaccacatcatccagatcagacaagatatagaccgtattaccaatga

SEQ ID NO: 2 - protein, 491 aa, Homo sapiens
MDHTDNELQGTNSSGSLGGLDVRRRIPIKLISKQANKAKPAPRTQRTINRMPAKAPPGDEEGFDYNEEERYDCKGGELFANQRRFPGHLFWDFQINILGEKDDTPVHFCDKCGLPIKIYGRMIPCKHVFCYDCAILHEKKGDKMCPGCSDPVQRIEQCTRGSLFMCSIVQGCKRTYLSQRDLQAHINHRHMRAGKPVTRASLENVHPPIAPPPTEIPERFIMPPDKHHMSHIPPKQHIMMPPPPLQHVPHEHYNQPHEDIRAPPAELSMAPPPPRSVSQETFRISTRKHSNLITVPIQDDSNSGAREPPPPAPAPAHHHPEYQGQPVVSHPHHIMPPQQHYAPPPPPPPPISHPMPHPPQAAGTPHLVYSQAPPPPMTSAPPPITPPPGHIIAQMPPYMNHPPPGPPPPQHGGPPVTAPPPHHYNPNSLPQFTEDQGTPSPPFTQPGGMSPGIWPAPRGPPPPPRLQGPPSQTPLPGPHHPDQTRYRPYYQ

SEQ ID NO: 3 - DNA, 25 bp, Homo sapiens
gggttattggtggtggagcagaggt

SEQ ID NO: 4 - DNA, 25 bp, Homo sapiens
tggagacgaggtggtggttattggg

SEQ ID NO: 5 - DNA, 21 bp, Homo sapiens
aagctcatctccaaacaagca